(19) United States
(12) Patent Application Publication
(10) Pub. No.: US 2026/0318894 A1
(43) Pub. Date: Oct. 15, 2026

(54) FOOD SPOILAGE MONITORING

(71) Applicant: Springhouse Technologies Inc., McLean, VA (US)

(72) Inventors: John Anderson, Brooklyn, NY (US); Boris Kontorovich, Brooklyn, NY (US); Tyler Sanborn, Queens, NY (US); Nick Fong, Brooklyn, NY (US)

(21) Appl. No.: 19/483,653

(22) PCT Filed: May 17, 2024
(86) PCT No.: PCT/US2024/029890

§ 371 Date: Nov. 12, 2025

Related U.S. Application Data
(60) Provisional application No. 63/467,449, filed on May 18, 2023.

(51) Int. Cl. G01N 33/02; F25D 29/00; G01N 33/00

(52) U.S. Cl. CPC G01N 33/02; F25D 29/008; G01N 33/004; G01N 33/0047; F25D 2400/22; F25D 2500/06

(57) ABSTRACT

Food spoilage monitoring may be achieved by measuring, using a sensor mounted to a storage compartment, a level of a gas within the storage compartment, generating a sensor signal indicative of the level of gas, starting a session in response to a drawer signal received by the session controller module, receiving the sensor signal over a first period of time and determining a baseline for the level of gas, receiving the sensor signal over a second period of time and determining an acceleration status indicative of whether the level of gas being produced within the storage compartment is accelerating or decelerating, determining a status of one or more items within the storage compartment based on the acceleration status, and generating a notification indicative of the status of one or more items within the storage compartment.

### CROSS-REFERENCE TO RELATED APPLICATIONS

This application claims the benefit of U.S. Provisional Patent Application, Ser. No. 63/467449 entitled “GAS SENSING AND MONITORING FOOD STORAGE STRUCTURE”, filed on May 18, 2023; the entirety of the above-noted application(s) is incorporated by reference herein.

### BACKGROUND

Foods that deteriorate and develop unpleasant odors, tastes, and textures are generally considered spoiled. Spoilage bacteria or fungus may cause fruits and vegetables to get mushy or slimy, or meat to develop a bad odor. Most people prefer not consume spoiled food. However, if accidentally consumed, people could become ill. Currently, there is no reliable way for consumers to detect when the produce stored in their refrigerators will begin to spoil, which results in significant waste. Consequently, there is demand for monitoring whether food is or is about to become spoiled.

### BRIEF DESCRIPTION

According to one aspect, a refrigerator for food spoilage monitoring may include a storage compartment, a sensor mounted to the storage compartment, and a computing device operably connected with the sensor. The sensor may be configured to measure a level of a gas within the storage compartment and generate a sensor signal indicative of the level of gas. The computing device may include a session controller module, a synchronizer module, and a monitoring module. The session controller module may be configured to start a session in response to a drawer signal received by the session controller module. The synchronizer module may be configured to receive the sensor signal over a first period of time and determine a baseline for the level of gas and receive the sensor signal over a second period of time and determine an acceleration status indicative of whether the level of gas being produced within the storage compartment is accelerating or decelerating. The monitoring module may be configured to determine a status of one or more items within the storage compartment based on the acceleration status.

According to one aspect, a refrigerator for food spoilage monitoring may include a storage compartment, an ethylene sensor mounted to the storage compartment, a carbon dioxide (CO₂) sensor mounted to the storage compartment, and a computing device operably connected with the ethylene sensor and the CO₂ sensor. The ethylene sensor may be configured to measure a level of ethylene gas within the storage compartment and generate an ethylene signal indicative of the level of ethylene gas. The CO₂ sensor may be configured to measure a level of CO₂ gas within the storage compartment and generate a CO₂ signal indicative of the level of CO₂ gas. The computing device may include a session controller module, a synchronizer module, and a monitoring module. The session controller module may be configured to start a session in response to a drawer signal received by the session controller module. The synchronizer module may be configured to receive the ethylene signal over a first period of time and determine a baseline for the level for the ethylene gas, receive the CO₂ signal over the first period of time and determine a baseline for the level of CO₂ gas, receive the ethylene signal over a second period of time and determine an ethylene acceleration status indicative of whether the level of ethylene gas being produced within the storage compartment is accelerating or decelerating, and receive the CO₂ signal over the second period of time and determine an CO₂ acceleration status indicative of whether the level of CO₂ gas being produced within the storage compartment is accelerating or decelerating. The monitoring module may be configured to determine a status of one or more items within the storage compartment based on the ethylene acceleration status and the CO₂ acceleration status.

According to one aspect, a computer-implemented method for food spoilage monitoring may include measuring, via a sensor mounted to a storage compartment, a level of a gas within the storage compartment and generating a sensor signal indicative of the level of gas, starting a session, via a processor, in response to a drawer signal, receiving, via the processor, the sensor signal over a first period of time and determining a baseline for the level of gas, receiving, via the processor, the sensor signal over a second period of time and determining an acceleration status indicative of whether the level of gas being produced within the storage compartment is accelerating or decelerating, determining, via the processor, a status of one or more items within the storage compartment based on the acceleration status, and generating, via the processor, a notification indicative of the status of one or more items within the storage compartment.

### BRIEF DESCRIPTION OF THE DRAWINGS

FIG. 1 is a block diagram illustrating a refrigerator including a food monitoring system.

FIG. 2 is a front view of the refrigerator with a door shown in an open position.

FIG. 3 is a perspective view of the refrigerator with the door shown in the open position.

FIG. 4 is a flow diagram of a computer-implemented method for food spoilage monitoring in the refrigerator.

FIG. 5 is an illustration of an example computer-readable medium or computer-readable device including processor-executable instructions configured to embody one or more of the provisions set forth herein, according to one aspect.

### DETAILED DESCRIPTION

The following includes definitions of selected terms employed herein. The definitions include various examples and/or forms of components that fall within the scope of a term and that may be used for implementation. The examples are not intended to be limiting. Further, one having ordinary skill in the art will appreciate that the components discussed herein, may be combined, omitted, or organized with other components or organized into different architectures.

A “processor”, as used herein, processes signals and performs general computing and arithmetic functions. Signals processed by the processor may include digital signals, data signals, computer instructions, processor instructions, messages, a bit, a bit stream, or other means that may be received, transmitted, and/or detected.

Generally, the processor may be a variety of various processors including multiple single and multicore processors and co-processors and other multiple single and multicore processor and co-processor architectures. The processor may include various modules to execute various functions.

A “memory”, as used herein, may include volatile memory and/or non-volatile memory. Non-volatile memory may include, for example, ROM (read only memory), PROM (programmable read only memory), EPROM (erasable PROM), and EEPROM (electrically erasable PROM). Volatile memory may include, for example, RAM (random access memory), synchronous RAM (SRAM), dynamic RAM (DRAM), synchronous DRAM (SDRAM), double data rate SDRAM (DDRSDRAM), and direct RAM bus RAM (DRRAM). The memory may store an operating system that controls or allocates resources of a computing device.

A “disk” or “drive”, as used herein, may be a magnetic disk drive, a solid state disk drive, a floppy disk drive, a tape drive, a Zip drive, a flash memory card, and/or a memory stick. Furthermore, the disk may be a CD-ROM (compact disk ROM), a CD recordable drive (CD-R drive), a CD rewritable drive (CD-RW drive), and/or a digital video ROM drive (DVD-ROM). The disk may store an operating system that controls or allocates resources of a computing device.

A “bus”, as used herein, refers to an interconnected architecture that is operably connected to other computer components inside a computer or between computers. The bus may transfer data between the computer components. The bus may be a memory bus, a memory controller, a peripheral bus, an external bus, a crossbar switch, and/or a local bus, among others. The bus may also be a vehicle bus that interconnects components inside a vehicle using protocols such as Media Oriented Systems Transport (MOST), Controller Area network (CAN), Local Interconnect Network (LIN), among others.

A “database”, as used herein, may refer to a table, a set of tables, and a set of data stores (e.g., disks) and/or methods for accessing and/or manipulating those data stores.

An “operable connection”, or a connection by which entities are “operably connected”, is one in which signals, physical communications, and/or logical communications may be sent and/or received. An operable connection may include a wireless interface, a physical interface, a data interface, and/or an electrical interface.

A “computer communication”, as used herein, refers to a communication between two or more computing devices (e.g., computer, personal digital assistant, cellular telephone, network device) and may be, for example, a network transfer, a file transfer, an applet transfer, an email, a hypertext transfer protocol (HTTP) transfer, and so on. A computer communication may occur across, for example, a wireless system (e.g., IEEE 802.11), an Ethernet system (e.g., IEEE 802.3), a token ring system (e.g., IEEE 802.5), a local area network (LAN), a wide area network (WAN), a point-to-point system, a circuit switching system, a packet switching system, among others.

A “mobile device”, as used herein, may be a computing device typically having a display screen with a user input (e.g., touch, keyboard) and a processor for computing. Mobile devices include handheld devices, portable electronic devices, smart phones, laptops, tablets, and e-readers.

### System Overview

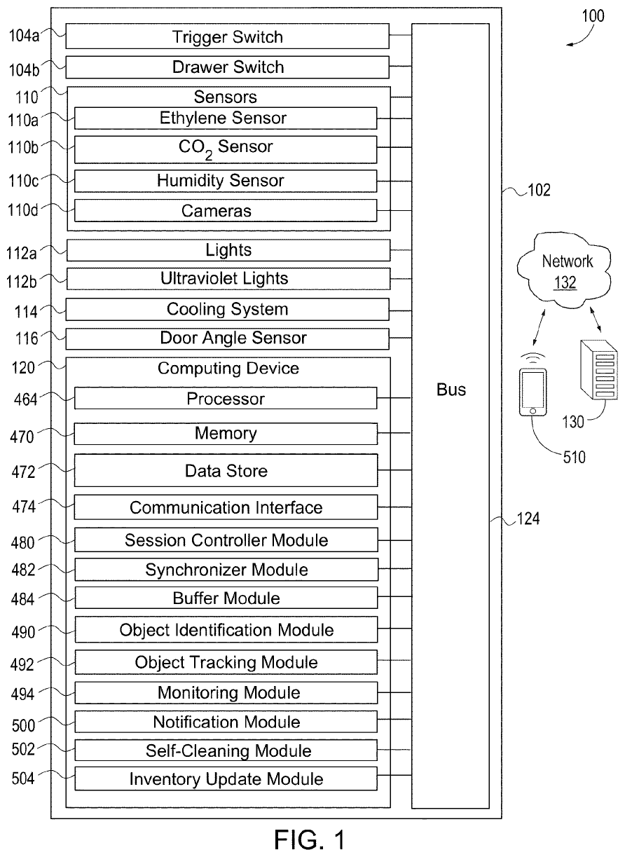

Referring now to the drawings, which are for purposes of illustrating one or more exemplary embodiments and not for purposes of limiting the same, FIG. 1 is a block diagram illustrating a refrigerator 102 including a food monitoring system. The refrigerator 102 may include one or more switches (e.g., a trigger switch 104a, a drawer switch 104b), one or more sensors 110 (e.g., an ethylene sensor 110a, a carbon dioxide (CO₂) sensor 110b, a humidity sensor 110c, cameras 110d, etc.), one or more lights (lights 112a, ultraviolet (UV) lights 112b), a cooling system 114, a door angle sensor 116 and a computing device 120.

Each of the trigger switch 104a, the drawer switch 104b, the plurality of sensors 110, the lights 112a-112b, the cooling system 114, the door angle sensor 116, and the computing device 120, including an operating system, may be operably connected by a bus 124. The components of the system 100, as well as the components of other systems, hardware architectures, and software architectures discussed herein, may be combined, omitted, or organized into different architectures for various embodiments.

The computing device 120 may be configured to control the lights 112a (depicted schematically in FIG. 1) during a session for processing by computer vision models described herein. The computing device 120 may control configuration aspects of cameras 110d including a frame rate and an exposure for optimized processing by the computer vision models.

The cooling system 114 may be similar to those found in known refrigerators, and therefore, are not described in greater detail herein. The cooling system 114 may include temperature sensors for sensing temperatures within the refrigerator 102 and air blower fans supplying air cooled by cooling devices (not shown), which may include a compressor (not shown) compressing refrigerant, a condenser (not shown) condensing the compressed refrigerant, an expansion valve (not shown) decompressing the condensed refrigerant, and an evaporator (not shown) evaporating the decompressed refrigerant, etc.

It will be appreciated that the computing device 120 may be implemented as a part of the refrigerator 102 or another device, e.g., a remote server 130 in computer communication via a network 132. In other words, the computing device 120 may be configured to provide wired or wireless computer communications utilizing various protocols to transmit and receive electronic signals internally to and from components of the system 100. Additionally, the computing device 120 may be operably connected to respective components for internal computer communication via the bus 124 (e.g., a Controller Area Network (CAN) or a Local Interconnect Network (LIN) protocol bus) to facilitate data input and output between the computing device 120 and the components of the system 100.

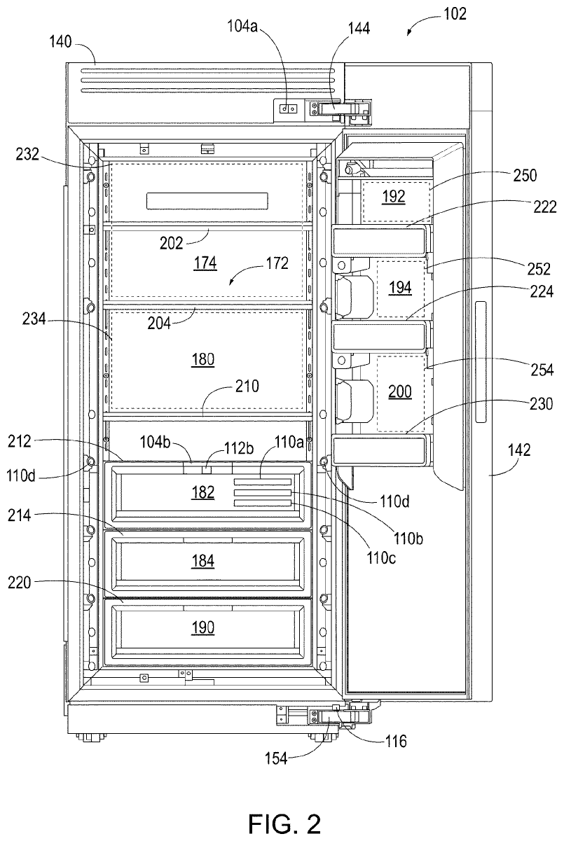

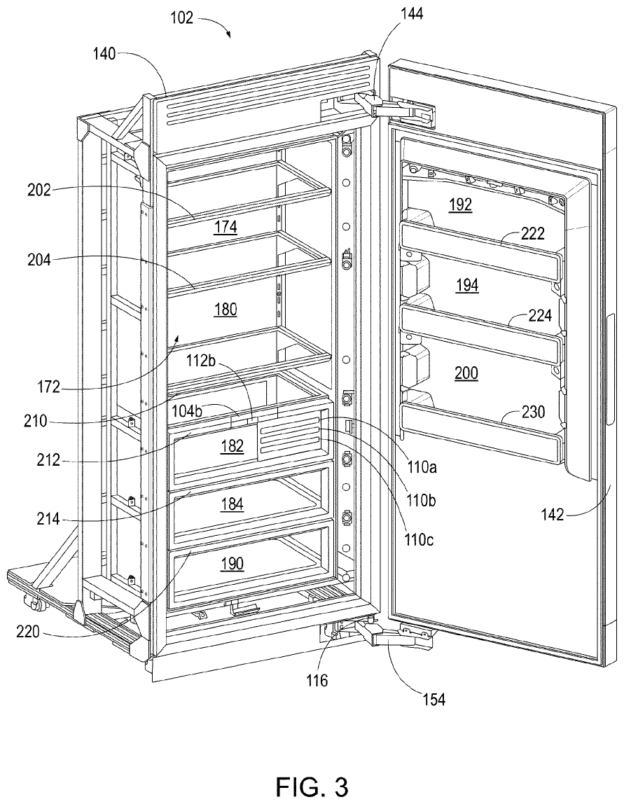

FIG. 2 is a front view of the refrigerator with a door shown in an open position. FIG. 3 is a perspective view of the refrigerator with the door shown in the open position. As shown in FIG. 2, the refrigerator 102 includes a main body 140 and a door 142 pivotally mounted to and movable with respect to the main body 140 between an open position and a closed position. The door 142 is connected or attached to the main body 140 through an upper hinge 144 and a lower hinge 154. The main body 140 and the door 142 define a plurality of storage compartments (e.g., food storage compartments) within a refrigerated enclosure 172 when the door 142 is in the closed position. The plurality of storage compartments includes a first, second, third, fourth, and fifth storage compartments 174, 180, 182, 184, 190, respectively, which are each located within the main body 140 in the illustrated embodiment. A sixth, seventh, and eighth storage compartment 192, 194, 200, respectively are located on the door 142 in the illustrated embodiment. It will be appreciated that the number and the location of the storage compartments 174, 180, 182, 184, 190, 192, 194, and 200 may differ from that shown in FIGS. 2-3.

As illustrated in FIGS. 2-3, a first shelf 202 and a second shelf 204 are disposed in the first storage compartment 174. A third shelf 210 is disposed in the second storage compartment 180. A first drawer 212 is disposed in the third storage compartment 182. A second drawer 214 is disposed in the fourth storage compartment 184. A third drawer 220 is disposed in the fifth storage compartment 190. A first bin 222 is disposed in the sixth storage compartment 192. A second bin 224 is disposed in the seventh storage compartment 194. A third bin 230 is disposed in the eighth storage compartment 200. It will be appreciated that the number and the location of the shelves 202, 204, 210, drawers 212, 214, 220, and bins 222, 224, 230 may differ from that shown in FIGS. 2-3.

Each storage compartment 174, 180, 182, 184, 190, 192, 194, and 200 defines a respective entrance opening through which items, such as food or produce, may be placed into and taken out of the corresponding storage compartment. For example, with reference to FIG. 2, the first storage compartment 174 defines a first entrance opening 232 leading onto the first shelf 202 and the second shelf 204, and the second storage compartment 180 defines a second entrance opening 234 leading onto the third shelf 210. The third storage compartment 182 defines a third entrance opening (not visible in FIGS. 2 and 3) leading into the first drawer 212, the fourth storage compartment 184 defines a fourth entrance opening (not visible in FIGS. 2 and 3) leading into the second drawer 214, and the fifth storage compartment 190 defines a fifth entrance opening (not visible in FIGS. 2 and 3) leading into the third drawer 220 when the respective drawers 212, 214, 220 are opened. The sixth storage compartment 192 defines a sixth entrance opening 250 leading into the first bin 222, the seventh storage compartment 194 defines a seventh entrance opening 252 leading into the second bin 224, and the eighth storage compartment 200 defines an eighth entrance opening 254 leading into the third bin 230.

With exemplary reference to the first drawer 212 disposed in the third storage compartment 182, the first drawer 212 may be equipped with one or more of the sensors 110, including the ethylene sensor 110a, the CO₂ sensor 110b, the humidity sensor 110c, and the cameras 110d such that one or more of the respective sensors are positioned or mounted to an interior portion of the first drawer 212 or the third storage compartment 182. However, the cameras 110d may be positioned above the first drawer 212 with a field of view encompassing the third entrance opening (not visible in FIGS. 2 and 3) leading into the first drawer 212 when the first drawer 212 is opened. In this manner, cameras 110d may be positioned to have a field of view that includes a respective entrance opening leading to the corresponding third storage compartment 182 when the door 142 is in the open position. In this way, the cameras 110d are configured to capture images of an item passing through any of the entrance openings as the food item is being loaded into or is being removed from the corresponding storage compartment.

Although merely the first drawer 212 and third storage compartment 182 are described as being equipped with one or more of the sensors 110, including the ethylene sensor 110a, the CO₂ sensor 110b, the humidity sensor 110c, and the cameras 110d, it will be appreciated that any of the storage compartments 174, 180, 182, 184, 190, 192, 194, 200, drawers 212, 214, 220, and bins 222, 224, 230, etc. may be configured or equipped with the respective sensors.

The sensors 110 may be configured to generate one or more sensor signals including sensor data which may be communicated to the computing device 120 via the bus 124. In this way, the sensors 110 may be operatively connected to the computing device 120. The computing device 120 may be configured to retrieve readings from any of the sensors 110 on a periodic basis. The gas sensors 110a, 110b may be configured to measure a level of a gas within a corresponding area (e.g., the third storage compartment 182, as described herein in the exemplary embodiment) and generate a sensor signal indicative of the level of gas. For example, the ethylene sensor 110a may be operatively connected to the computing device 120 and configured to measure a level of ethylene gas within the storage compartment, generate an ethylene signal indicative of the level of ethylene gas, and transmit the ethylene signal to the computing device 120 periodically. The CO₂ sensor 110b may be operatively connected to the computing device 120 and configured to measure a level of CO₂ gas within the storage compartment, generate a CO₂ signal indicative of the level of CO₂ gas, and transmit a CO₂ signal to the computing device 120 periodically. The humidity sensor 110c may be configured to measure a level of humidity near a location of the humidity sensor 110c, generate, and transmit a humidity signal to the computing device 120. The cameras 110d may be configured to capture one or more images and transmit the images to the computing device 120. Respective sensor signals or sensor data may be processed by the computing device 120, as will be described in greater detail herein.

With reference to FIGS. 1-2, the trigger switch 104a is mounted to the main body 140, is operably connected with the computing device 120, and is configured to detect a position of the door 142. In other words, the trigger switch 104a determines whether the door 142 is in the open position or the closed position. The trigger switch 104a is configured to send a trigger signal to the computing device 120 based on whether the door 142 is in the open position or the closed position. For example, the trigger switch 104a is configured to send a stop trigger signal to the computing device 120 when the door 142 moves from the closed position toward the open position, and the trigger switch 104a is configured to send a start trigger signal to the computing device 120 when the door 142 moves from the open position to the closed position. The trigger switch 104a may include at least one of various types of position sensors such as mechanical switches, potentiometers, piezoelectric sensors, Hall effect sensors, or eddy-current sensors without departing from the scope of the present disclosure. Also, while the trigger switch 104a is shown disposed on the main body 140, the trigger switch 104a may additionally or alternatively be positioned on the door 142.

The drawer switch 104b is mounted to the first drawer 212, is operably connected with the computing device 120, and is configured to detect a position of the first drawer 212. In other words, the drawer switch 104b determines whether the first drawer 212 is in the open position or the closed position. The drawer switch 104b is configured to send a drawer signal to the computing device 120 indicative of whether the first drawer 212 is in the open position or the closed position. For example, the drawer switch 104b is configured to send a stop drawer signal to the computing device 120 when the first drawer 212 moves from the closed position toward the open position, and the drawer switch 104b is configured to send a start drawer signal to the computing device 120 when the first drawer 212 moves from the open position to the closed position. The drawer switch 104b may include at least one of various types of position sensors such as mechanical switches, potentiometers, piezoelectric sensors, Hall effect sensors, or eddy-current sensors without departing from the scope of the present disclosure. Also, while the drawer switch 104b is shown disposed on the first drawer 212, the drawer switch 104b may additionally or alternatively be positioned on the main body 140, the third storage compartment 182, etc.

The door angle sensor 116 is configured to determine the angle of the door 142 with respect to the main body 140. The door angle sensor 116, for example, may include a rotary encoder having a shaft that rotates as the door 142 pivots with respect to the main body 140 to output an angle signal indicative of the relative angular position of the door 142 with respect to the main body 140 and communicate the angle signal to the computing device 120.

With reference again to FIG. 1, the computing device 120 includes a processor 464, a memory 470, a data store 472, and a communication interface 474, which are each operably connected for computer communication via the bus 124. The data store 472 may house or store a machine learning model or a tree-based model trained during a training phase. These models may be utilized to interpret sensor data received by the computing device 120, and are discussed in greater detail herein. The communication interface 474 provides software and hardware to facilitate data input and output between the components of the computing device 120 and other components, networks, and data sources, also described in greater detail herein. The computing device 120 also includes a session controller module 480, a synchronizer module 482, a buffer module 484, an object identification module 490, an object tracking module 492, a monitoring module 494, a notification module 500, a self-cleaning module 502, and an inventory update module 504 for monitoring freshness of objects, produce, food, or inventory in the refrigerator 102. Any of the aforementioned modules 480, 482, 484, 490, 492, 494, 500, 502, 504, etc. may be implemented via the processor 464, the memory 470, the data store 472, etc.

### Description of Modules

The session controller module 480 may be configured to start a session in response to a start trigger signal, a start drawer signal, or a signal from the door angle sensor 116 received by the session controller module 480. In this way, the session controller module 480 may be configured to start a session for a corresponding zone (e.g., associated with a storage compartment, bin, enclosure, etc.) in response to a corresponding trigger signal or drawer signal associated with that zone received by the session controller module 480 by assigning a session identification (ID) to the started session. Any sensor data received by the computing device 120 during the started session is automatically associated with the session ID. The session controller module 480 may be configured to retrieve readings from any of the sensors 110 (e.g., including sensors 110a-110d) on a periodic basis and assign timestamps to respective readings for respective sensor signals. It will be appreciated that different zones or enclosed areas (e.g., storage compartments 174, 180, 182, 184, 190, 192, 194, 200, drawers 212, 214, 220, bins 222, 224, 230, refrigerated enclosure 172, etc.) have their own sessions.

As discussed above, the third storage compartment 182 is described as the exemplary embodiment and is equipped with the ethylene sensor 110a, the CO₂ sensor 110b, the humidity sensor 110c, and the cameras 110d. However, any of the zones or enclosed areas of the refrigerator 102 may be outfitted or equipped with one or more of the sensors 110, including sensors 110a-110d.

The synchronizer module 482 may be configured to receive the sensor signal over a first period of time and determine a baseline for the level of gas during the first period of time. For example, the synchronizer module 482 may receive the ethylene signal over the first period of time and determine the baseline for the level of ethylene gas and receive the CO₂ signal over the first period of time and determine the baseline for the level of CO₂ gas. According to one example, the baseline may be set by the synchronizer module 482 when the corresponding zone receives no change in the trigger signal or the drawer signal (e.g., a start trigger signal changing to a stop trigger signal or vice-versa, a start drawer signal changing to a stop drawer signal or vice-versa, etc.) associated with that zone for a predetermined period of time. For example, with respect to the third storage compartment 182, the baseline may be set or determined when the drawer switch 104b remains undisturbed for a period of time greater than six hours, eight hours, or overnight, etc. The reason the baseline is set over the predetermined period of time is because ripening of produce or food items, as it pertains to CO₂, is generally stable. However, for individual produce or food items, total CO₂ emission is generally arbitrary. Therefore, tracking may be determined by acceleration or deceleration of the baseline level for respective gasses.

The synchronizer module 482 may be configured to receive the sensor signal over a second period of time and determine an acceleration status indicative of whether the level of gas being produced within the storage compartment is accelerating or decelerating. The synchronizer module 482 may determine the acceleration status based on the machine learning model or the tree based model stored in the data store by feeding the sensor data to the models to generate an interpretation of the sensor data. In this way, the models may predict, adjust, and notify a user based on the determined baseline and determine an acceleration threshold for determining the acceleration status.

The synchronizer module 482 may receive the ethylene signal over a second period of time (e.g., after the first period of time when the baseline is being determined or set) and determine an ethylene acceleration status indicative of whether the level of ethylene gas being produced within the storage compartment is accelerating or decelerating and receive the CO₂ signal over the second period of time and determine an CO₂ acceleration status indicative of whether the level of CO₂ gas being produced within the storage compartment is accelerating or decelerating. In this way, once the baseline is set, the synchronizer module 482 may monitor the sensor signals (e.g., the ethylene signal or the CO₂ signal) to observe changes or deviations from the baseline, and generate a corresponding acceleration status indicative of the change.

The object identification module 490 may be configured to identify one or more items within the storage compartment or identify a number of items within the storage compartment. The object identification module 490 is in operable connection with the cameras 110d. After the session is started or initialized, sensor data received by the computing device 120 is automatically associated with the session ID and the session controller module 480 may instruct the cameras 110d to begin capturing images based on the start trigger signal received by the session controller module 480 from the trigger switch 104a. The cameras 110d begin capturing images after the session controller module 480 receives the start drawer signal from the drawer switch 104b, and the cameras 110d cease capturing images after the session controller module 480 receives the stop drawer signal from the drawer switch 104b. The cameras 110d are each configured to compress captured images, assign the session ID, assign a camera ID, and assign a frame ID for each captured image and send the compressed images along with their respective session ID, camera ID and frame ID to the synchronizer module 482, which may occur during or after the session (e.g., the time between opening and closing of the door 142). A session may include the transferring of multiple items into and out of the refrigerator 102 and associated storage compartments.

The buffer module 484 may be configured to store compressed captured images as an image buffer memory and may be configured to send the compressed captured images to a remote server, (e.g., the remote server 130 of FIG. 1), for post processing after the session ends. With this configuration, system resources of the computing device 120 may be directed to image capture during the session, and then directed to post processing when the session has ended, thereby reducing a capacity of the computing device 120 required for receiving image information from the cameras 110d and tracking inventory in the refrigerator 102.

The object identification module 490 is configured to determine whether each captured image in a respective frame set contains an object, to define a bounding box around each object located in the respective captured image and to assign a class identification to the object. Based on the number of bounding boxes, the object identification module 490 may determine a number of items or food items within a storage compartment, bin, or enclosure. Further, this information may be timestamped and associated with the session ID. Class identifications assigned by the object identification module 490 indicate an inventory type of the object. The inventory type may be maintained in the memory 470 and/or data store 472 of the computing device 120, or stored remotely and accessed via the network 132. The inventory type may indicate a category of ingredient, food product, meal, dish, or other object type maintained in the database.

The object identification module 490 includes a computer vision model that outputs a bounding box around the object and the class identification for the object for each captured image. In an embodiment, the computer vision model is YOLOv5, however the object identification module 490 may include additional or alternative computer vision models for determining object presence and class identification in the refrigerator 102 without departing from the scope of the present disclosure.

The computer vision model used in the object identification module 490 may also be configured to remove images from frame sets received from the synchronizer module 482 that do not contain an object and to output an updated group of images to the object tracking module 492. The updated group of images, which may include only captured images that contain an object, may include a lesser number of images as compared to the frame set received by the object identification module 490 from the synchronizer module 482. The first captured image that contains an object in the updated group of images may indicate the start of a sequence, which indicates a hand motion in or out of the refrigerator. The last captured image that contains an object in the updated group of images may indicate the end of the sequence. Where the object identification module 490 identifies no object, for example the object identification module 490 identifies an empty hand without any object to be loaded into or removed from the refrigerator, the last captured image containing the object may indicate the end of the sequence. Each sequence, or detected motion in or out of the refrigerator 102, may contain multiple objects. For example, a person may insert two items (e.g., an apple and a cup of yogurt) into or out of the refrigerator in a single sequence. In such an instance, the object identification module 490 assigns a first class identification to the apple that appears as one object in the captured image and assigns a second class identification to the cup of yogurt that appears as another object in the captured image.

The object tracking module 492 is configured to analyze sequential captured images from each camera of the cameras 110d tracking objects among the sequential captured images per camera. In an embodiment, the object tracking module 492 compares a first n images captured in the updated group of images during a sequence to a last n images captured in the updated group of images during the sequence to track movement of objects during the sequence and/or a change in the number of objects for the corresponding storage compartment. Determination of the temporal sequence of captured images is based on the frame ID associated with each image. For example, captured images having a higher frame ID may be captured later in time as compared to those with a lower frame ID. Determination of the camera that captured a respective captured image is based on the camera ID associated with the respective captured image. In this regard, the object tracking module 492 associates an object with itself across sequential images per camera, thereby tracking movement of the object over time.

In an embodiment, the object tracking module 492 is also configured to assign a track ID to associate the object in one analyzed image to the object in other analyzed images in the cameras 110d. Track IDs assigned by the object tracking module 492 are unique to each camera of the cameras 110d, and unique to each object captured by the cameras 110d in sequential captured images. With reference to the apple and cup of yogurt example discussed above, the object tracking module 492 is configured to assign a different track ID to the object that appears as the apple in the captured image than the track ID for the object that appears as the cup of yogurt in the captured image. Also, the track ID for the object that appears as the apple in the captured image from camera 260a of the first set of cameras 260a, 260b will be a different track ID for the object that appears as the apple in the captured image from camera 260b. In this manner, each track ID assigned by the object tracking module 492 identifies a single object across sequential captured images by a single camera in the cameras 110d.

The object tracking module 492 may employ an observation-centric simple online and real-time tracking (OCSORT) algorithm for analyzing the sequential captured images from each camera of the cameras 110d. With this configuration, the object tracking module 492 receives the updated group of images with bounding boxes and class IDs from the object identification module 490 and the OCSORT algorithm may process the updated group of images, and then output a further updated group of images with the bounding boxes, class IDs and additionally include associated track IDs for each object located in each captured image in the further updated group of images.

The inventory update module 504 is configured to communicate with the memory 470 and/or the data store 472 via the bus 124 and/or the remote server 130 via the network 132 to update an inventory status of at least one storage compartment in the plurality of storage compartments based on each inventory change and the direction assigned thereto. The inventory update module 504 may also be configured to send all captured images of a session to the remote server 130 via a data pipeline routed through the network 132. The inventory update module 504 may also be configured to package corresponding subsequence data including object identification information, track ID information, and direction information for each merge identification, and to send the subsequence data to the object storage service with the captured images.

When an object is detected as going out of the refrigerator 102 by the object identification module 490, then the food item that was located in the refrigerator and was the object associated with that particular item, is removed from the inventory of the refrigerator 102. This inventory information may be stored in the memory 470, the data store 472, and/or the remote server 130 and may be output to an application accessible by a mobile device 510 for viewing the inventory information on a display of the mobile device 510 on from the notification module 500.

The session controller module 480 may be configured to start a second session in response to a change in the trigger signal and/or drawer signal over a third period of time and the object identification module 490 identifying one or more different items within the storage compartment over the third period of time. In other words, the session controller module 480 may start the second session when the drawer or door to the corresponding zone (e.g., storage compartment) is opened and new items are added to the storage compartment.

The session controller module 480 may be configured to continue the session in response to a change in the trigger signal and/or drawer signal over a third period of time and the object identification module 490 identifying at least some of the one or more items within the storage compartment over the third period of time and identifying a fewer number of items within the storage compartment over the third period of time. In other words, the session controller module 480 may continue the session (e.g., rather than starting the second session) when the drawer or door to the corresponding zone (e.g., storage compartment) is opened and food items are merely removed from the storage compartment, for example.

In either scenario, the object identification module 490 may facilitate confirmation of whether new items are added to the storage compartment or items are removed from the storage compartment based on the inventory tracking or using the computer vision model.

The monitoring module 494 may be configured to determine a status of one or more items within the storage compartment based on the acceleration status (e.g., the ethylene acceleration status and the CO₂ acceleration status). Generally, as produce or food items progress through the ripening and spoiling process, three changes in the baseline levels may occur, such as an acceleration of ethylene gas indicating that produce is ripening, a deceleration of ethylene gas indicating that produce is already ripened, an acceleration of CO₂ gas indicating that produce has begun to spoil. Therefore, the monitoring module 494 may determine the status of one or more of the food items to be a ripening status in response to the ethylene acceleration status indicative of the level of ethylene gas accelerating, an already ripened status in response to the ethylene acceleration status indicative of the level of ethylene gas decelerating, or a spoiled status in response to the CO₂ acceleration status indicative of the level of CO₂ gas accelerating.

According to an example where there is merely a single gas sensor, the monitoring module 494 may determine the status of one or more of the food items to be a ripening status in response to the sensor being the ethylene sensor and the acceleration status indicative of the level of ethylene gas accelerating, an already ripened status in response to the sensor being the ethylene sensor and the acceleration status indicative of the level of ethylene gas decelerating, or a spoiled status in response to the sensor being the CO₂ sensor and the acceleration status indicative of the level of CO₂ gas accelerating.

According to one example, the monitoring module 494 may be configured to determine the status of one or more items within the relevant storage compartment based on the acceleration status, the baseline for the level of gas, and the machine learning model or the tree-based model. For example, during the training phase, the machine learning model or the tree-based model may be trained to identify threshold acceleration and deceleration levels indicative of different statuses of produce based on the baseline levels of corresponding gases. In other words, depending on the level of the initial baseline for a given gas, the model may predict different statuses for different levels of gas acceleration or deceleration. The reason for this is that different types of produce may emit different amounts of ethylene gas during ripening and different amounts of CO₂ gas during spoilage. Therefore, training of the model enables the system 100 to more accurately identify when the current payload or food items are transitioning from one phase or state to another (e.g., unripened, ripening, already ripened, spoiling, spoiled, etc.). According to one example, the machine learning model or the tree-based model may be trained using training data including a ground-truth dataset during the training phase, transmitted to the system 100, and stored on the data store 472.

The notification module 500 may be configured to generate a notification indicative of the status of one or more items within the corresponding storage compartment. Examples of the status include the ripening status, the already ripened status, the spoiled status, etc. When the food item is associated with the ripening status or the already ripened status, the monitoring module 494 may utilize the machine learning model or the tree-based model from the data store 472 to calculate an estimated time until the food item reaches a spoiled status. When the food item is associated with the spoiling or spoiled status, the notification may be generated to indicate to the user that the food has spoiled. The notification module 500 may transmit the notification to the communication interface 474, and may be output to an application accessible by a mobile device 510 across the network 132 for viewing the inventory information on a display of the mobile device 510.

The UV lights 112b may, for example, be mounted to the storage compartment. The self-cleaning module 502 may be configured to activate the UV lights 112b in response to the monitoring module 494 determining the status of one or more of the food items to be the spoiled status. Further, if the self-cleaning module 502 has performed UV disinfection, the notification module 500 may generate a notification indicating the same to the user.

### Method Overview

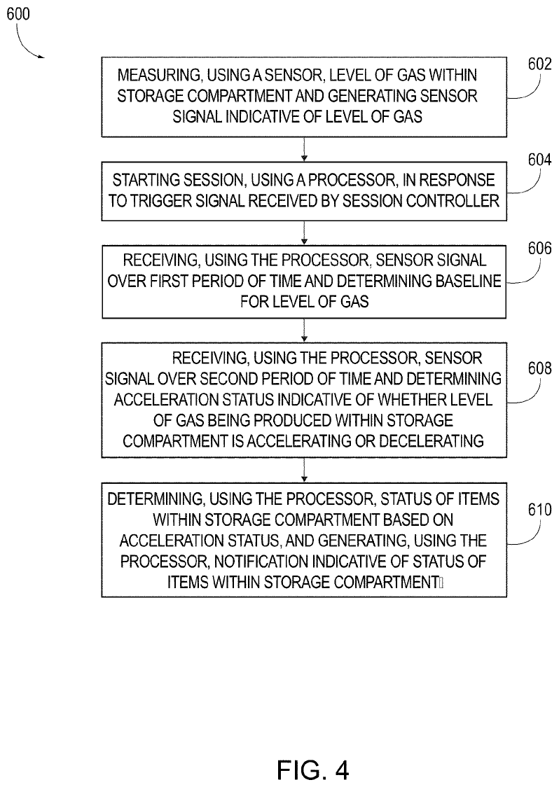

FIG. 4 is a flow diagram of a computer-implemented method 600 for food spoilage monitoring in the refrigerator. The computer-implemented method 600 for food spoilage monitoring may include measuring 602, via a sensor mounted to a storage compartment, a level of a gas within the storage compartment and generating a sensor signal indicative of the level of gas, starting 604 a session, via a processor, in response to a start trigger signal received by the session controller, receiving 606, via the processor, the sensor signal over a first period of time and determining a baseline for the level of gas, receiving 608, via the processor, the sensor signal over a second period of time and determining an acceleration status indicative of whether the level of gas being produced within the storage compartment is accelerating or decelerating, determining 610, via the processor, a status of one or more items within the storage compartment based on the acceleration status, and generating, via the processor, a notification indicative of the status of one or more items within the storage compartment.

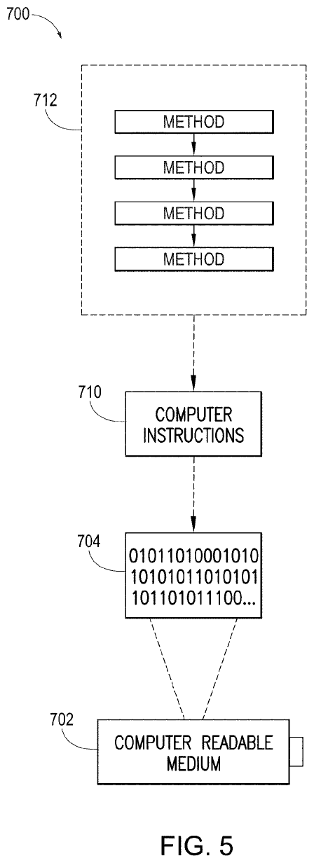

Still another aspect involves a computer-readable medium including processor-executable instructions configured to implement one aspect of the techniques presented herein. An aspect of a computer-readable medium or a computer-readable device devised in these ways is illustrated in FIG. 5, wherein an implementation 700 includes a computer-readable medium 702, such as a CD-R, DVD-R, flash drive, a platter of a hard disk drive, etc., on which is encoded computer-readable data 704. This encoded computer-readable data 704, such as binary data including a plurality of zero's and one's as shown in 704, in turn includes a set of processor-executable computer instructions 710 configured to operate according to one or more of the principles set forth herein. In this implementation 700, the processor-executable computer instructions 710 may be configured to perform a method 712, such as the computer-implemented method 600 of FIG. 4. In another aspect, the processor-executable computer instructions 710 may be configured to implement a system, such as the system 100 of FIG. 1. Many such computer-readable media may be devised by those of ordinary skill in the art that are configured to operate in accordance with the techniques presented herein.

As used in this application, the terms “component”, “module,” “system”, “interface”, and the like are generally intended to refer to a computer-related entity, either hardware, a combination of hardware and software, software, or software in execution. For example, a component may be, but is not limited to being, a process running on a processor, a processing unit, an object, an executable, a thread of execution, a program, or a computer. By way of illustration, both an application running on a controller and the controller may be a component. One or more components residing within a process or thread of execution and a component may be localized on one computer or distributed between two or more computers.

Further, the claimed subject matter is implemented as a method, apparatus, or article of manufacture using standard programming or engineering techniques to produce software, firmware, hardware, or any combination thereof to control a computer to implement the disclosed subject matter. The term “article of manufacture” as used herein is intended to encompass a computer program accessible from any computer-readable device, carrier, or media. Of course, many modifications may be made to this configuration without departing from the scope or spirit of the claimed subject matter.

Although the subject matter has been described in language specific to structural features or methodological acts, it is to be understood that the subject matter of the appended claims is not necessarily limited to the specific features or acts described above. Rather, the specific features and acts described above are disclosed as example aspects.

Various operations of aspects are provided herein. The order in which one or more or all of the operations are described should not be construed as to imply that these operations are necessarily order dependent. Alternative ordering will be appreciated based on this description. Further, not all operations may necessarily be present in each aspect provided herein.

As used in this application, “or” is intended to mean an inclusive “or” rather than an exclusive “or”. Further, an inclusive “or” may include any combination thereof (e.g., A, B, or any combination thereof). In addition, “a” and “an” as used in this application are generally construed to mean “one or more” unless specified otherwise or clear from context to be directed to a singular form. Additionally, at least one of A and B and/or the like generally means A or B or both A and B. Further, to the extent that “includes”, “having”, “has”, “with”, or variants thereof are used in either the detailed description or the claims, such terms are intended to be inclusive in a manner similar to the term “comprising”.

Further, unless specified otherwise, “first”, “second”, or the like are not intended to imply a temporal aspect, a spatial aspect, an ordering, etc. Rather, such terms are merely used as identifiers, names, etc. for features, elements, items, etc. For example, a first channel and a second channel generally correspond to channel A and channel B or two different or two identical channels or the same channel. Additionally, “comprising”, “comprises”, “including”, “includes”, or the like generally means comprising or including, but not limited to.

It will be appreciated that various of the above-disclosed and other features and functions, or alternatives or varieties thereof, may be desirably combined into many other different systems or applications. Also, that various presently unforeseen or unanticipated alternatives, modifications, variations, or improvements therein may be subsequently made by those skilled in the art which are also intended to be encompassed by the following claims.

1. A refrigerator for food spoilage monitoring, comprising:
a storage compartment;
a sensor mounted to the storage compartment configured to measure a level of a gas within the storage compartment and generate a sensor signal indicative of the level of gas; and
a computing device operably connected with the sensor, the computing device including:
a session controller module configured to start a session in response to a drawer signal received by the session controller module;
a synchronizer module configured to:
receive the sensor signal over a first period of time and determine a baseline for the level of gas; and
receive the sensor signal over a second period of time and determine an acceleration status indicative of whether the level of gas being produced within the storage compartment is accelerating or decelerating; and
a monitoring module configured to determine a status of one or more items within the storage compartment based on the acceleration status.

2. The refrigerator for food spoilage monitoring of claim 1, wherein the sensor is an ethylene sensor or a carbon dioxide (CO₂) sensor.

3. The refrigerator for food spoilage monitoring of claim 2, wherein the monitoring module determines the status of one or more of the items to be:
a ripening status in response to the sensor being the ethylene sensor and the acceleration status indicative of the level of gas accelerating;
an already ripened status in response to the sensor being the ethylene sensor and the acceleration status indicative of the level of gas decelerating; or
a spoiled status in response to the sensor being the CO₂ sensor and the acceleration status indicative of the level of gas accelerating.

4. The refrigerator for food spoilage monitoring of claim 3, comprising an ultraviolet (UV) light mounted to the storage compartment, wherein the computing device includes a self-cleaning module configured to activate the UV light in response to the monitoring module determining the status of one or more of the items to be the spoiled status.

5. The refrigerator for food spoilage monitoring of claim 1, wherein the computing device includes a notification module configured to generate a notification indicative of the status of one or more items within the storage compartment.

6. The refrigerator for food spoilage monitoring of claim 1, wherein the monitoring module is configured to determine the status of one or more items within the storage compartment based on the acceleration status, the baseline for the level of gas, and a machine learning model or a tree-based model.

7. The refrigerator for food spoilage monitoring of claim 6, wherein the status of one or more items within the storage compartment is indicative of an estimated time until one or more items reaches a spoiled status.

8. The refrigerator for food spoilage monitoring of claim 1, comprising a camera configured to capture one or more images of the one or more items within the storage compartment, wherein the computing device includes an object identification module configured to identify the one or more items within the storage compartment or identify a number of items within the storage compartment.

9. The refrigerator for food spoilage monitoring of claim 8, wherein the session controller module is configured to start a second session in response to a change in the drawer signal over a third period of time and the object identification module identifying one or more different items within the storage compartment over the third period of time.

10. The refrigerator for food spoilage monitoring of claim 8, wherein the session controller module is configured to continue the session in response to a change in the drawer signal over a third period of time and the object identification module identifying at least some of the one or more items within the storage compartment over the third period of time and identifying a fewer number of items within the storage compartment over the third period of time.

11. A refrigerator for food spoilage monitoring, comprising:
a storage compartment;
an ethylene sensor mounted to the storage compartment configured to measure a level of ethylene gas within the storage compartment and generate an ethylene signal indicative of the level of ethylene gas;
a carbon dioxide (CO₂) sensor mounted to the storage compartment configured to measure a level of CO₂ gas within the storage compartment and generate a CO₂ signal indicative of the level of CO₂ gas; and
a computing device operably connected with the ethylene sensor and the CO₂ sensor, the computing device including:
a session controller module configured to start a session in response to a drawer signal received by the session controller module;
a synchronizer module configured to:
receive the ethylene signal over a first period of time and determine a baseline for the level for the ethylene gas;
receive the CO₂ signal over the first period of time and determine a baseline for the level of CO₂ gas;
receive the ethylene signal over a second period of time and determine an ethylene acceleration status indicative of whether the level of ethylene gas being produced within the storage compartment is accelerating or decelerating; and
receive the CO₂ signal over the second period of time and determine an CO₂ acceleration status indicative of whether the level of CO₂ gas being produced within the storage compartment is accelerating or decelerating; and
a monitoring module configured to determine a status of one or more items within the storage compartment based on the ethylene acceleration status and the CO₂ acceleration status.

12. The refrigerator for food spoilage monitoring of claim 11, wherein the monitoring module determines the status of one or more of the items to be:
a ripening status in response to the ethylene acceleration status indicative of the level of ethylene gas accelerating;
an already ripened status in response to the ethylene acceleration status indicative of the level of ethylene gas decelerating; or
a spoiled status in response to the CO₂ acceleration status indicative of the level of CO₂ gas accelerating.

13. The refrigerator for food spoilage monitoring of claim 12, comprising an ultraviolet (UV) light mounted to the storage compartment, wherein the computing device includes a self-cleaning module configured to activate the UV light in response to the monitoring module determining the status of one or more of the items to be the spoiled status.

14. The refrigerator for food spoilage monitoring of claim 11, wherein the computing device includes a notification module configured to generate a notification indicative of the status of one or more items within the storage compartment.

15. The refrigerator for food spoilage monitoring of claim 11, wherein the monitoring module is configured to determine the status of one or more items within the storage compartment based on the CO₂ acceleration status and the ethylene acceleration status, the baseline for the level of gas, and a machine learning model or a tree-based model.

16. The refrigerator for food spoilage monitoring of claim 15, wherein the status of one or more items within the storage compartment is indicative of an estimated time until one or more items reaches a spoiled status.

17. The refrigerator for food spoilage monitoring of claim 11, comprising a camera configured to capture one or more images of the one or more items within the storage compartment, wherein the computing device includes an object identification module configured to identify the one or more items within the storage compartment or identify a number of items within the storage compartment.

18. The refrigerator for food spoilage monitoring of claim 17, wherein the session controller module is configured to start a second session in response to a change in the drawer signal over a third period of time and the object identification module identifying one or more different items within the storage compartment over the third period of time.

19. The refrigerator for food spoilage monitoring of claim 17, wherein the session controller module is configured to continue the session in response to a change in the drawer signal over a third period of time and the object identification module identifying at least some of the one or more items within the storage compartment over the third period of time and identifying a fewer number of items within the storage compartment over the third period of time.

20. A computer-implemented method for food spoilage monitoring, comprising:
measuring, via a sensor mounted to a storage compartment, a level of a gas within the storage compartment and generating a sensor signal indicative of the level of gas;
starting a session, via a processor, in response to a drawer signal;
receiving, via the processor, the sensor signal over a first period of time and determining a baseline for the level of gas;
receiving, via the processor, the sensor signal over a second period of time and determining an acceleration status indicative of whether the level of gas being produced within the storage compartment is accelerating or decelerating;
determining, via the processor, a status of one or more items within the storage compartment based on the acceleration status; and
generating, via the processor, a notification indicative of the status of one or more items within the storage compartment.
